(19) United States
(12) Patent Application Publication
(10) Pub. No.: US 2022/0108779 A1
(43) Pub. Date: Apr. 7, 2022

(54) REHABILITATION ASSISTANCE SYSTEM, REHABILITATION ASSISTANCE METHOD, AND PROGRAM

(71) Applicant: TOYOTA JIDOSHA KABUSHIKI KAISHA, Toyota-shi Aichi-ken (JP)

(72) Inventors: Takeru Fukagawa, Chiryu-shi (JP); Issei Nakashima, Toyota-shi Aichi-ken (JP); Hisataka Yuasa, Nagoya-shi Aichi-ken (JP); Takuya Iwata, Toyota-shi Aichi-ken (JP); Takashi Shimizu, Nisshin-shi (JP); Yohei Otaka, Kariya-shi Aichi-ken (JP); Satoshi Hirano, Nagoya-shi Aichi-ken (JP); Masaki Katoh, Nagoya-shi Aichi-ken (JP); Daisuke Imoto, Nagoya-shi (JP); Shin Kitamura, Nagoya-shi (JP)

(73) Assignee: TOYOTA JIDOSHA KABUSHIKI KAISHA, Toyota-shi Aichi-ken (JP)

(21) Appl. No.: 17/486,171

(22) Filed: Sep. 27, 2021

(30) Foreign Application Priority Data
Oct. 2, 2020 (JP) 2020-168111

(51) Int. Cl. G16H 15/00; G16H 50/70; G16H 10/60; G06F 16/23; A61B 5/00

(52) U.S. Cl. CPC G16H 15/00; G16H 50/70; G16H 20/30; G06F 16/2379; A61B 5/4848; G16H 10/60

(57) ABSTRACT

A rehabilitation assistance system, a rehabilitation assistance method, and a program capable of appropriately assisting rehabilitation are provided. A rehabilitation assistance system according to an embodiment includes: a storage unit configured to store, for each rehabilitation patient, evaluation indices of evaluation items regarding the evaluation items for evaluating a level of achievement of rehabilitation training; a processing unit configured to divide a plurality of rehabilitation patients into groups in accordance with the evaluation indices; and a display unit configured to update a display that shows a state of progress of a target patient in accordance with progress information of rehabilitation training and show the display.

### CROSS REFERENCE TO RELATED APPLICATIONS

This application is based upon and claims the benefit of priority from Japanese patent application No. 2020-168111, filed on Oct. 2, 2020, the disclosure of which is incorporated herein in its entirety by reference.

### BACKGROUND

The present disclosure relates to a rehabilitation assistance system, a rehabilitation assistance method, and a program.

Patent Literature 1 (Japanese Unexamined Patent Application Publication No. 2018-79308) discloses a rehabilitation assistance apparatus that changes a mode of training in accordance with a level of achievement of a rehabilitation exercise and updates the mode of training.

### SUMMARY

There has been a demand, when the above rehabilitation is performed, to assist the rehabilitation more appropriately.

The present disclosure has been made in view of the aforementioned circumstances, and aims to provide a rehabilitation assistance system, a rehabilitation assistance method, and a program capable of appropriately assisting rehabilitation.

A rehabilitation assistance system according to one embodiment of the present disclosure includes: a database configured to store, for each rehabilitation patient, evaluation indices of evaluation items regarding the evaluation items for evaluating a level of achievement of rehabilitation training; a processing unit configured to divide a plurality of rehabilitation patients into groups in accordance with the evaluation indices; and a display unit configured to update a display that shows a state of progress of a target patient in one of the groups in accordance with progress information of rehabilitation training and show the display.

The above rehabilitation assistance system may display information on the number of persons indicating the number of rehabilitation patients who belong to the group as the display that shows the state of progress of the target patient.

In the above rehabilitation assistance system, two or three evaluation items among a plurality of evaluation items may be set as priority evaluation items, a plurality of rehabilitation patients who belong to the selected group may be divided into subgroups in accordance with the evaluation indices of non-priority evaluation items other than the priority evaluation items, and information on the number of persons indicating the number of rehabilitation patients who belong to the subgroup may be displayed.

In the above rehabilitation assistance system, the number of priority evaluation items may be two and the information on the number of persons of the group may be displayed in a form of a two-dimensional matrix.

The above rehabilitation assistance system may include filtering the plurality of rehabilitation patients according to a search condition in accordance with the target patient.

In the above rehabilitation assistance system, the evaluation items may include the FIM.

In the above rehabilitation assistance system, the evaluation items may include adaptive equipment worn by the rehabilitation patient.

In the above rehabilitation assistance system, the evaluation items may include an assistive device used by the rehabilitation patient.

A rehabilitation assistance method according to this embodiment includes: referring to a database configured to store, for each rehabilitation patient, evaluation indices of evaluation items regarding the evaluation items for evaluating a level of achievement of rehabilitation training, and dividing a plurality of rehabilitation patients into groups; and updating a display that shows a state of progress of a target patient in one of the groups in accordance with progress information of rehabilitation training and showing the display.

The above rehabilitation assistance system may include displaying information on the number of persons indicating the number of rehabilitation patients who belong to the group as the display that shows the state of progress of the target patient.

In the above rehabilitation assistance system, two or three evaluation items among a plurality of evaluation items may be set as priority evaluation items, a plurality of rehabilitation patients who belong to the selected group may be divided into subgroups in accordance with the evaluation indices of non-priority evaluation items other than the priority evaluation items, and information on the number of persons indicating the number of rehabilitation patients who belong to the subgroup may be displayed.

In the above rehabilitation assistance method, the number of priority evaluation items may be two, and the information on the number of persons of the group may be displayed in a form of a two-dimensional matrix.

The above rehabilitation assistance method may include filtering the plurality of rehabilitation patients according to a search condition in accordance with the target patient.

In the above rehabilitation assistance method, the evaluation items may include the FIM.

In the above rehabilitation assistance method, the evaluation items may include adaptive equipment worn by the rehabilitation patient.

In the above rehabilitation assistance method, the evaluation items may include an assistive device used by the rehabilitation patient.

A program according to this embodiment causes a computer to execute the above rehabilitation assistance method.

The aim of the present disclosure is to provide a rehabilitation assistance system, a rehabilitation assistance method, and a program capable of appropriately assisting rehabilitation.

The above and other objects, features and advantages of the present disclosure will become more fully understood from the detailed description given hereinbelow and the accompanying drawings which are given by way of illustration only, and thus are not to be considered as limiting the present disclosure.

### BRIEF DESCRIPTION OF DRAWINGS

FIG. 1 is a block diagram showing a configuration of a rehabilitation training system;

FIG. 2 is a table showing one example of data collected by a rehabilitation assistance apparatus;

FIG. 3 is a table showing one example of data collected by the rehabilitation assistance apparatus;

FIG. 4 is a diagram showing one example of a display screen of the rehabilitation assistance apparatus; and

FIG. 5 is a flowchart showing a method in the rehabilitation assistance apparatus.

### DESCRIPTION OF EMBODIMENTS

Hereinafter, the present disclosure will be explained with reference to an embodiment of the present disclosure. However, the disclosure set forth in the claims is not limited to the following embodiment. For a purpose of clarifying the description, the following description and the drawings will be omitted and simplified as appropriate. Throughout the drawings, the same components are denoted by the same reference symbols and overlapping descriptions will be omitted as necessary.

### EMBODIMENTS

Hereinafter, with reference to the drawings, an embodiment of the present disclosure will be described. A rehabilitation training system according to this embodiment performs processing for enabling a trainee to carry out one or a plurality of kinds of training for the purpose of recovering functions of his/her physical actions. The trainee here is, for example, a person suffering from paralysis in a part of his/her body (this person is also called a rehabilitation patient), which is caused by a stroke (cerebrovascular disease) or a spinal cord injury. The trainee's physical action functions have been impaired due to the above disease. In this embodiment, physical actions are, for example, actions included in Activities of Daily Living (ADL) and include actions such as eating, toileting, dressing, walking, etc.

A trainee carries out one or a plurality of kinds of training in order to recover the functions of his/her impaired physical actions. To be specific, for example, training for recovering functions of a physical action eating includes training of a meal intake action, training of a swallowing action, and the like. Further, the training for recovering functions of the toileting action includes, for example, training of a moving action for moving to each of a bed, a wheelchair, and a toilet seat, training for toileting, and the like. Further, the training for recovering the function of the walking action includes, for example, training for maintaining the sitting posture, training for maintaining the standing posture, training of flat ground walking, training of walking on a slope, and the like. Further, the training includes, besides the training in which the trainee actively carries out physical actions as described above, passive training such as a massage in which the trainee receives stimulation to his/her joints or muscles from a helper. The aforementioned training is also referred to as rehabilitation or a rehabilitation training.

Some training performed using the rehabilitation training system may require assistance by therapists. Therapists are persons who have expertise that is necessary to assist trainees. In this embodiment, the therapists include a physical therapist, an occupational therapist, and a speech therapist. The therapists assist training performed by the trainees in accordance with expertise that they each have.

The physical therapist is a person who offers a physical therapy for persons who have physical disabilities mainly for the purpose of recovering fundamental action abilities. The physical therapy is a therapeutic treatment performed on persons whose functions of physical actions have been impaired due to diseases, injuries, disabilities and the like for the purpose of maintaining or improving these functions. The physical therapist carries out a physical therapy by using exercises, heat, electricity and the like. The physical therapist is also referred to as a PT.

The occupational therapist is a person who offers an occupational therapy for persons who have physical or mental disabilities mainly for the purpose of recovering their applicative action abilities or social adaptability. The “occupation” here includes daily activities such as bathing, dressing, and toileting. The occupational therapist is also referred to as an OT.

The speech therapist is a person who carries out language training and other training exercises and provides tests, advice, guidance, and other assistance necessary for the above training exercises for persons who have disabilities in their speech functions, language functions, or hearing for the purpose of maintaining and improving these functions. The speech therapist further conducts training such as meal intake or swallowing under the instructions of doctors or dentists. The speech therapist is also referred to as a speech-language-hearing therapist or an ST. Hereinafter, persons such as the PT, the OT, and the ST who assist rehabilitation training performed by trainees are also called assistants. The assistants may include doctors, nurses and so on.

The rehabilitation training system helps an assistant set a goal for a trainee. For example, the rehabilitation training system is a rehabilitation assistance apparatus (this apparatus is also referred to as a rehabilitation assistance system) that collects data regarding rehabilitation training conducted by a plurality of trainees. Then the assistance apparatus refers to data of past rehabilitation patients and displays a display screen for enabling rehabilitation to be assisted. A display unit of the rehabilitation assistance apparatus displays a screen for enabling the goal for the trainee (target patient) to be set. For example, the assistant and the trainee are able to set a goal to be achieved at a predetermining timing such as at the time of discharge while looking at the screen. The assistant or the trainee is able to make a rehabilitation plan in accordance with the goal.

### First Embodiment

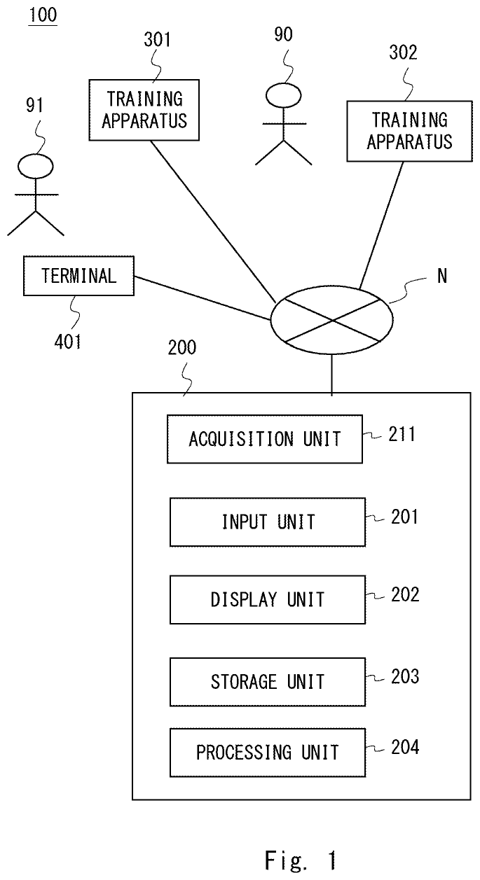

With reference to FIG. 1, an example of a rehabilitation training system (this system is simply referred to as a training system) will be described. A training system 100 includes an assistance apparatus 200, training apparatuses 301 and 302, and a terminal 401. While the training system 100 includes two training apparatuses 301 and 302 in FIG. 1, the training system 100 may instead include only one training apparatus 301. Further, a plurality of rehabilitation patients 90 may perform training using the training apparatuses 301 and 302.

It is assumed here that the training apparatuses 301 and 302 are training equipment for providing walking training. That is, the rehabilitation patient 90 performs rehabilitation training for improving his/her walking functions using the training apparatuses 301 and 302. The training apparatuses 301 and 302 are, for example, Welwalk WW-1000 or WW-2000. As a matter of course, the rehabilitation training is not limited to walking training and may instead be training of contacting or toileting. The training apparatuses 301 and 302 include drive equipment such as a motor, or a sensor. The training apparatuses 301 and 302 are installed in a rehabilitation facility of a hospital or the like. As a matter of course, the training apparatuses 301 and 302 may be equipment installed in the house of the rehabilitation patient 90. While only one rehabilitation patient 90 is shown in FIG. 1, a plurality of rehabilitation patients 90 may perform rehabilitation training.

The terminal 401 is a device that enables an assistant 91 or the like to input data. The terminal 401 is, for example, a tablet computer, a smartphone, a personal computer or the like. The assistant 91 is able to input data regarding the rehabilitation patient 90 or rehabilitation training performed by the rehabilitation patient 90 using the terminal 401. The terminal 401 may be a device attached to the training apparatus 301 or the training apparatus 302. The rehabilitation training system 100 may include a plurality of terminals 401. As described above, the assistant 91 is a PT, an OT, an ST, a doctor, or a nurse.

The assistance apparatus 200 is a device to/from which data can be input/output. The assistance apparatus 200 includes an input unit 201, a display unit 202, a storage unit 203, a processing unit 204, and an acquisition unit 211. The assistance apparatus 200 may be, like the terminal 401, a tablet computer, a smartphone, a personal computer or the like. The assistance apparatus 200 may use some of the functions of the terminal 401. The assistance apparatus 200 serves as a rehabilitation assistance system that assists rehabilitation training.

The acquisition unit 211 acquires data from the training apparatuses 301 and 302 and the terminal 401. The training apparatuses 301 and 302, the terminal 401, and the assistance apparatus 200 are connected to one another in such a way that they can communicate with one another via a network N. The network N is a communication line network such as the Internet, an intranet, a mobile telephone network, or a Local Area Network (LAN).

The acquisition of data by the acquisition unit 211 is not limited to the one that uses the network N. For example, at least a part of data may be acquired via a USB memory or the like. The data may be acquired by wireless communication such as WiFi (registered trademark) or by wired communication. The acquisition unit 211 includes a wireless or wired data input/output interface. The data acquisition may be automatically performed by the acquisition unit 211. The assistant 91 may select necessary data and write the data into the assistance apparatus 200.

The storage unit 203 stores various kinds of data acquired by the acquisition unit 211. The storage unit 203 includes a non-volatile memory such as a hard disc drive, a flash memory, an Erasable Programmable Read Only Memory (EPROM) or a Solid State Drive (SSD).

The input unit 201 accepts an input from the assistant 91 or the rehabilitation patient 90. The input unit 201 includes an input device such as a touch panel, a keyboard, a mouse or the like. Furthermore, a voice input using a microphone may be performed.

The display unit 202 displays a predetermined screen for the assistant 91 or the rehabilitation patient 90. The display unit 202 includes, for example, a display device such as a liquid crystal display. The display unit 202 may further include a display control circuit or the like of the display device.

The processing unit 204 refers to the data stored in the storage unit 203 and divides past rehabilitation patients into groups. The processing of the processing unit 204 will be described later.

The assistance apparatus 200 is not limited to a physically single apparatus. For example, the display unit 202 and the input unit 201 may serve as a main body apparatus of a tablet PC or the like and the storage unit 203 may serve as a storage device that is provided separately from the main body apparatus. Then the tablet PC may acquire the data from the storage unit 203 using a wireless LAN such as WiFi (registered trademark).

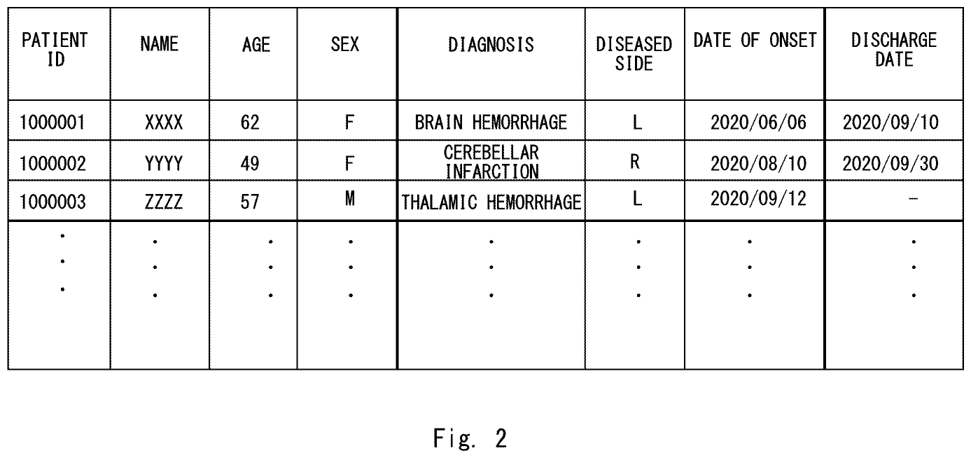

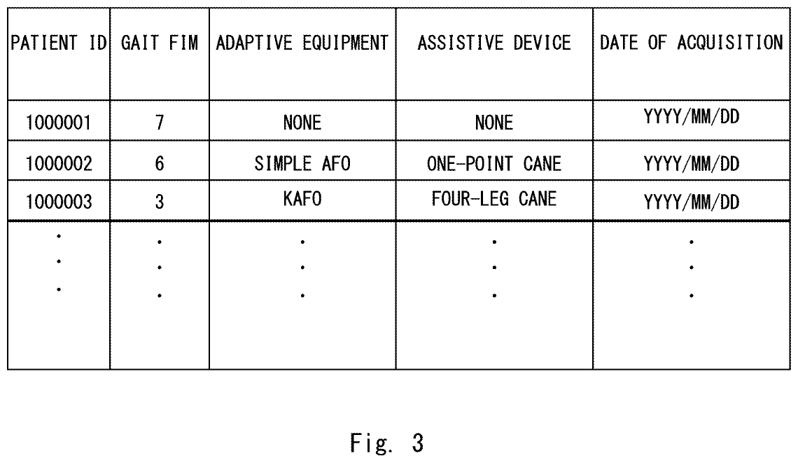

The storage unit 203 includes a database that stores information regarding a plurality of rehabilitation patients. That is, the storage unit 203 stores, for each rehabilitation patient, information regarding the rehabilitation patient. With reference to FIGS. 2 and 3, one example of the data stored in the storage unit 203 will be described. FIG. 2 is a table showing a list of patient information regarding the rehabilitation patient.

As shown in FIG. 2, the storage unit 203 stores the patient information for each patient as a database. The patient information indicates, for example, the attributes etc. of the rehabilitation patient 90. For example, data such as the patient ID, the age, the sex, the diagnosis, the diseased side, the date of the onset of symptoms, and the discharge date is stored as the patient information. The diseased side, which is data indicating the diseased leg that is paralyzed, is shown by a left leg (L) or a right leg (R). Regarding the rehabilitation patients that have not yet been discharged, that is, the rehabilitation patients who are still in the hospital, the discharge date is left blank (patient ID 1000003 in FIG. 2). In other words, it is possible to identify whether the rehabilitation patient is still in the hospital or has already been discharged by checking whether the discharge date is input.

The patient information is stored in the storage unit 203 by, for example, the assistant 91 inputting it from the terminal 401. Further, the assistant 91 may be able to check the patient information by inputting the patient ID or the name through the terminal 401. Further, when the assistance apparatus 200 acquires data from a plurality of hospitals or facilities, a facility ID indicating a hospital or a facility where the rehabilitation training has been performed may be added.

As a matter of course, the items shown in FIG. 2 are examples of the patient information and some of the items may be omitted. Further, items that are not shown in FIG. 2 may be added. Specifically, data of higher-order brain functions, the current FIM, the current adaptive equipment, the current assistive device, and a suspending device may be added to the patient ID. Further, the patient information may include the physical size (body height, weight, etc.), the Br. stage, the SIAS, the initial gait FIM, the latest gait FIM, etc. The initial gait FIM is the gait FIM at the time of the onset of symptoms or before rehabilitation training is started. The patient information may include various kinds of data indicating the physical ability of the rehabilitation patient 90.

The Br. stage, which indicates Brunnstrom Recovery stage, divides the process of recovery from hemiplegia into six stages based on observation. Of the Br. stage, the data may contain the items in the lower extremity section, which are main items related to the walking training apparatus. The SIAS stands for Stroke Impairment Assessment Set, which is an index for comprehensively evaluating the dysfunction caused by a stroke. The SIAS may contain hip-flexion test (Hip-Flex), knee-extension test (Knee-Ext), and foot-pat test (Foot-Pat). The SIAS may also contain lower extremity light touch (Touch L/E), lower extremity position sense (Position L/E), abdominal muscle strength (Abdominal), and verticality test (Verticality).

The Functional Independence Measure (FIM) defines one of evaluation methods for Activities of Daily Life (ADL). In the FIM, each item is rated on a 7-point scale from one point to seven points based on the level of assistance.

FIG. 3 is a table showing evaluation items for evaluating the level of recovery of the rehabilitation patient 90. Since it is assumed, in this example, that walking training is performed, three evaluation items, namely, the gait FIM, the assistive device, and the adaptive equipment, are set. An evaluation index indicating the level of recovery is set for each of the evaluation items. That is, the storage unit 203 stores the evaluation indices indicating the level of recovery for each of the rehabilitation patients 90.

For example, the gait FIM serves as a general index indicating the level of recovery. Further, the gait FIM is an index indicating the action ability (i.e., walking ability) of the trainee when an actuator is not used. When a trainee is able to walk 50 m or more without being helped by a helper and without a harness (assistive device), the highest number of points, that is, seven points are counted. When the trainee can barely walk less than 15 m no matter how hard one helper assists him/her, the lowest number of points, that is, one point is counted. Further, when the trainee is able to move 50 m with a minimum amount of assistance (amount of assistance: 25% or less), the number of points counted is four. When the trainee is able to move 50 m with a moderate level of assistance (amount of assistance: 25% or more), the number of points counted is three. Accordingly, as the recovery progresses, the gait FIM of the rehabilitation patient 90 is gradually increased.

The evaluation index of the gait FIM is indicated by seven levels since it is input using integers from one to seven. The evaluation index of the gait FIM may instead be indicated by eight levels, including a case in which no data is input (i.e., blank).

The adaptive equipment complements or replaces the physical functions of the rehabilitation patient 90 and is used continuously over a long period of time. The rehabilitation patient 90 walks wearing the adaptive equipment. The item of the adaptive equipment is indicated by nine types, namely, none, others, a simple Ankle Foot Orthoses (AFO), Remodeled Adjustable Posterior Strut (RAPS)-AFO, M (Molded)-AFO, Knee Ankle Foot Orthoses (KAFO), and blank. That is, the evaluation item of the adaptive equipment is categorized into nine evaluation indices in accordance with the level of recovery.

The assistive device is equipment for assisting walking by the rehabilitation patient 90. The rehabilitation patient 90 walks without wearing an assistive device. The rehabilitation patient 90 walks holding an assistive device such as a cane. The evaluation item of the assistive device is indicated by nine types, namely, none, others, a one-point cane, a four-point cane (small), a four-point cane (large), a four-leg cane, a walker, parallel bars, and blank.

The rehabilitation patient 90 is able to walk using an appropriate assistive device and appropriate adaptive equipment. Further, the assistant 91 is able to recommend an appropriate assistive device and an appropriate adaptive equipment to the rehabilitation patient 90 in accordance with the walking functions of the rehabilitation patient 90. The assistive device and the adaptive equipment that are used are gradually changed in accordance with the level of recovery of the rehabilitation patient 90. In other words, the adaptive equipment and the assistive device are evaluation indices for evaluating the level of recovery of the rehabilitation patient 90.

The storage unit 203 stores, for each patient, the evaluation indices of all the evaluation items. As a matter of course, regarding some evaluation items, evaluation indices may be blank. The evaluation indices are updated in accordance with the state of recovery of the rehabilitation patient 90. That is, the storage unit 203 stores the latest evaluation indices. The evaluation indices are associated with the date of the acquisition when the data has been acquired. The evaluation indices may be associated with the number of days passed since the date of the onset of symptoms. The storage unit 203 may store, besides the date of acquisition, the time of the acquisition etc.

The storage unit 203 may store initial (at the time of the onset of symptoms) evaluation indices or may store evaluation indices for each predetermined period of time after the date of the onset of symptoms. The storage unit 203 may store evaluation indices in accordance with the number of days since the rehabilitation is started in time series. The evaluation indices of the evaluation items may be input using the terminal 401 or may be automatically acquired by the training apparatuses 301 and 302.

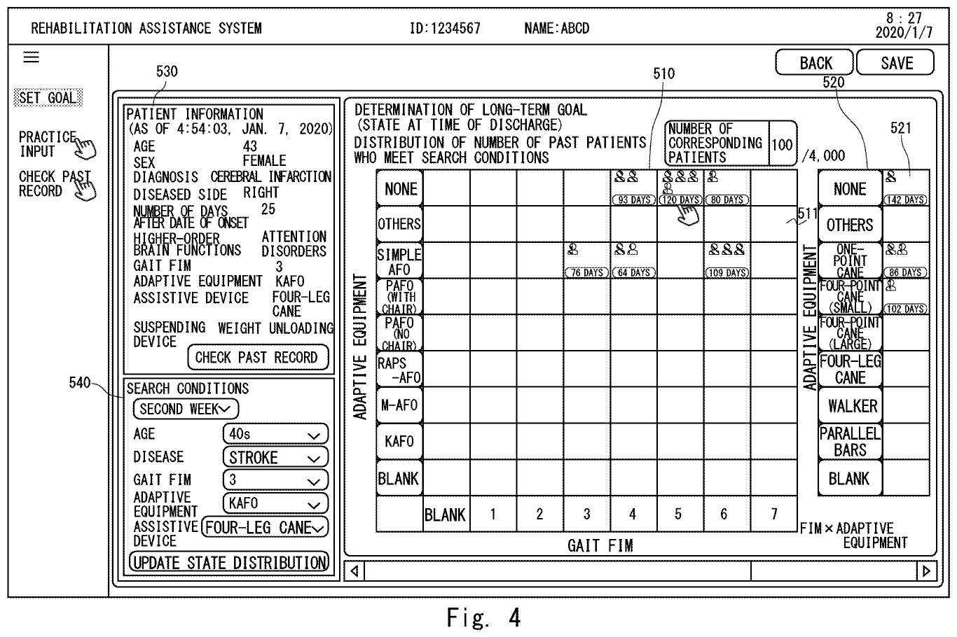

FIG. 4 is a diagram showing one example of a display screen displayed on the display unit 202. FIG. 4 shows a display screen for enabling the assistant 91 or the rehabilitation patient 90 to set a goal of rehabilitation. In this example, the rehabilitation goal is set by the assistant 91 or the rehabilitation patient 90 by referring to information on past rehabilitation patients.

The display screen includes a first display region 510, a second display region 520, a patient information display region 530, and a search condition display region 540. The patient information display region 530 displays patient information regarding the rehabilitation patient (this patient is also referred to as a target patient) for which the goal is set. The patient information display region 530 is, for example, a window that displays the age, the sex, the diagnosis, the diseased side and the like of the target patient.

The search condition display region 540 displays search conditions for looking for a patient who is similar to the target patient. That is, the search condition display region 540 is a window for enabling the assistant 91 to input the search conditions. The search condition display region 540 displays pull-down menus for enabling the assistant 91 to input search conditions.

In FIG. 4, the age (forties), the disease (stroke), the gait FIM (3), the adaptive equipment (KAFO), the assistive device (four-leg cane) and the like are set as the search conditions. The data of the search conditions is input in accordance with the information on the target patient. Further, the data of the search conditions may be automatically input from the information on the target patient or may be manually input by the assistant 91.

Further, the number of days elapsed since the onset of symptoms (second week) is set as one of the search conditions. It is possible to extract rehabilitation patients similar to the target patient by referring, for example, to data of each rehabilitation patient in the second week from the start of rehabilitation. By clicking a state distribution update distribution button, which is a search button, the assistance apparatus 200 refers to the database in the storage unit 203 and extracts rehabilitation patients who meet the search conditions. Accordingly, the past patients who are similar to the target patient are extracted from among the past rehabilitation patients (these patients are also called past patients). It is possible to filter the past patients according to the search conditions in accordance with the target patient. It is therefore possible to set the past patients with preconditions similar to those of the target patient as a population.

The first display region 510 displays a state of progress of the target patient so that the assistant 91 or the target patient can grasp the state of progress of the target patient. Further, the display in the first display region 510 is updated in accordance with the progress information of the rehabilitation training. For example, the first display region 510 displays information indicating the states of the past patients at the time of discharge who have met the search conditions in order to set the goal at the time of discharge.

Specifically, the processing unit 204 divides a plurality of past patients into groups in accordance with the states of past patients similar to the target patient at the time of discharge. That is, a plurality of past patients are divided into a plurality of groups. Then the first display region 510 indicates the distribution of the numbers of persons who belong to the respective groups. In this example, the first display region 510 displays the information on the number of past patients who meet the search conditions in a form of a two-dimensional matrix.

The two-dimensional matrix is generated in accordance with the evaluation indices of the evaluation items. Specifically, the horizontal axis of the two-dimensional matrix indicates the gait FIM at the time of discharge and the vertical axis indicates the adaptive equipment at the time of discharge. The evaluation items used to generate the two-dimensional matrix in the first display region 510 are set as priority evaluation items. That is, the gait FIM and the adaptive equipment are priority evaluation items. The evaluation items other than the priority evaluation items are non-priority evaluation items. The non-priority items include the assistive device. Whether one evaluation item is a priority evaluation item or a non-priority evaluation item is set in advance. That is, the evaluation items that are regarded to be important and indicate the recovery in the rehabilitation training are set as the priority evaluation items. That is, the evaluation items with a high contribution rate to the recovery are set as the priority evaluation items.

The gait FIM is divided into eight levels, including blank, and the adaptive equipment is divided into nine types. Since the past patients are divided into 72 (=8×9) groups, 72 display frames 511 are shown in the first display region 510 in FIG. 4. Each display frame 511 has a rectangular frame.

The display frames 511 display information on the numbers of persons indicating the numbers of past patients who belong to the respective groups. In this example, the number of persons is indicated by the number of icons, the sizes of the icons and the like. For example, one icon indicates 100 persons. Fractions of less than 100 persons are indicated by the size of the icon or the like. A display frame 511 with no icon indicates a group with no past patients similar to the target patient. As a matter of course, the information on the number of persons is not limited to the number of icons and the sizes of the icons. The information on the number of persons may be indicated by display color or color shades. The display unit 202 may set a color bar in accordance with the number of persons and change the display color in accordance with the increase/decrease in the number of persons. Alternatively, the display unit 202 may change grayscale shades depending on the number of persons. Further, the display unit 202 may display the number indicating the number of persons in the display frame 511 as the information on the number of persons.

The display frame 511 may further display information on the number of days in accordance with the number of days until discharge. That is, the average value or the median value of the number of days it took for a plurality of past patients who belong to the respective groups to be discharged is the information on the number of days. The processing unit 204 is able to obtain the information on the number of days from the date of admission and the date of discharge of each rehabilitation patient. The processing unit 204 refers to the database in the storage unit 203 and calculates the information on the number of persons and the information on the number of days. Whether or not one patient has been discharged can be determined in accordance with the presence or the absence of the data of the discharge date shown in FIG. 2. Then the evaluation indices on the acquisition date that is just before the discharge date are the evaluation indices at the time of discharge.

As described above, information indicating the current or future state of progress of the target patient is displayed in the group. Further, the display unit 202 updates and displays information in accordance with the progress of the rehabilitation. The assistant 91 or the target patient is able to easily grasp the degree of recovery of past patients similar to the target patient at the time of discharge. Further, the assistant 91 or the target patient is able to grasp how many days from the date of admission to the hospital it took for the past patients similar to the target patient to be discharged from the hospital. The assistant 91 is therefore able to make an appropriate plan for the target patient. That is, the assistant 91 is able to set the goal of the rehabilitation training by referring to the recovery processes of the past rehabilitation patients. Thus the target patient is able to maintain high motivation for the rehabilitation training.

The assistant 91 or the target patient is able to select a target group. When the assistant 91 clicks the display frame 511 of the group with the largest number of persons or the group with a relatively large number of persons, this group is set as the selected group. Alternatively, a group according to the level which the target patient desires to reach may be specified as the selected group. The assistant 91 is able to refer to a past rehabilitation history of the past patients who belong to the selected group and make a rehabilitation plan regarding the target patient. The assistant 91 is able to set a goal based on the comparison between the target patient and the past patients similar to the target patient.

Further, a distribution of the number of past patients who belong to the selected group is displayed in the second display region 520. In this example, the processing unit 204 divides the past patients in the selected group into subgroups based on the evaluation indices of the assistive device, which is a non-priority evaluation item.

Specifically, in the assistive device, the evaluation index is divided into nine types. Therefore, nine display frames 521 are shown in one line in the second display region 520. Each display frame 521 displays information on the number of past patients who belong to a subgroup and information on the number of days. Since the information on the number of persons and the information on the number of days displayed on the display frame 521 are the same as the information on the display frame 511, the descriptions thereof will be omitted.

Further, the assistant 91 or the target patient is able to select a target subgroup. When the assistant 91 clicks the display frame 521 of a subgroup with the largest number of persons or a subgroup with a relatively large number of persons, this subgroup is set as a selected subgroup.

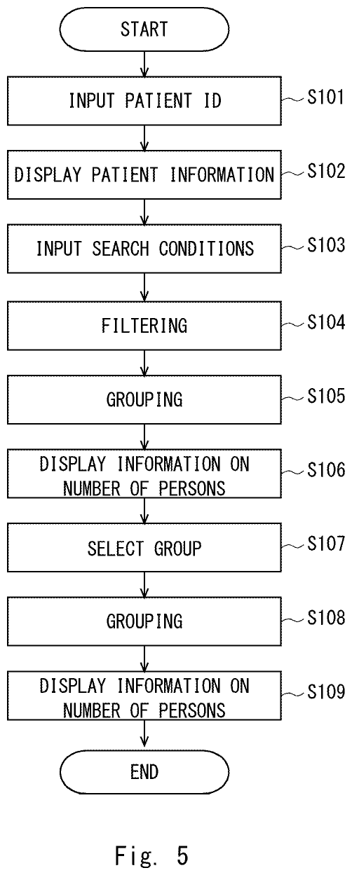

FIG. 5 is a flowchart showing a rehabilitation assistance method according to this embodiment. First, the assistant 91 inputs the patient ID of the target patient (S101). Then the display unit 202 displays information on the target patient (S102). For example, information on the target patient is displayed in the patient information display region 530 in FIG. 4. As a matter of course, the assistant 91 may input the patient information.

Next, the assistant 91 inputs the search conditions indicated in the search condition display region 540 (S103). For example, when the assistant 91 operates the input unit 201, the search conditions are input. As a matter of course, it is sufficient that the number of search conditions be one or more. Further, if it is desired to look up all the past patients as a population, Step S103 may be omitted. Some of the search conditions may be automatically input.

The processing unit 204 filters the past patients in accordance with the search conditions (S104). Then the processing unit 204 divides the past patients who meet the search conditions into groups (S105). That is, the processing unit 204 filters a plurality of past patients in accordance with the input search conditions. For example, past patients whose evaluation indices of two priority evaluation items match each other are categorized in one group. The display unit 202 displays information on the numbers of persons in the respective groups in a form of a two-dimensional matrix (S106). In this example, the display unit 202 may display, besides information on the number of persons, the information on the number of days.

After the assistant 91 selects the target selected group (S107), the processing unit 204 divides the past patients into subgroups (S108). That is, a plurality of past patients who belong to the selected group are divided into subgroups in accordance with evaluation indices of non-priority evaluation items. Then the display unit 202 displays the information on the number of persons for each subgroup (S109).

While the number of priority evaluation items is two, it may be three. In this case, the information on the number of persons may be displayed by changing the shades or the color of a three-dimensional graph. Further, it is sufficient that priority orders for evaluation be set in the priority evaluation items. The priority orders may be assigned to all the evaluation items in advance. The goal may be set for every two or three evaluation items.

Further, when there are a large number of evaluation items, the selection of a target group may be repeated. In this example, a case in which rehabilitation training regarding swallowing is performed will be described. In the swallowing rehabilitation, for example, five evaluation items, namely, swallowing Eating Status Scale (ESS), swallowing Dysphagia severity scale (DSS), water, normal diet (side dish), and posture, are set, which are prioritized in this order.

The processing unit 204 divides past patients into groups, with the swallowing ESS (Eating Status Scale) and the swallowing DSS being first priority evaluation items. The display unit 202 displays information on the number of persons in a form of a two-dimensional matrix in which the horizontal axis indicates the swallowing ESS and the vertical axis indicates the swallowing DSS.

After the assistant 91 selects one group, the processing unit 204 divides the past patients into groups, with water and the normal diet (side dish) being second priority evaluation items. The display unit 202 displays information on the number of persons in a form of a two-dimensional matrix in which the horizontal axis indicates water and the vertical axis indicates normal diet (side dish).

After the assistant 91 selects one group from the second matrix display, the processing unit 204 divides the past patients into groups, with posture being a third priority evaluation item. In this example, since the third priority evaluation item is one axis (one dimensional), the information on the number of persons is displayed in one line.

By repeating the grouping and the two-dimensional matrix display in this way, it is possible to appropriately assist the rehabilitation even when there are four or more evaluation items. That is, even when there are four or more evaluation items, priorities may be assigned to the evaluation items. Steps S107-S109 in FIG. 5 may be repeatedly performed in accordance with the number of evaluation items.

Since the assistant 91 is able to appropriately refer to the data of past patients, the assistant 91 is able to make a plan regarding the efficient rehabilitation. The assistant 91 is able to provide appropriate advice. Further, the target patient may perform rehabilitation training with high motivation.

As described above, the storage unit 203 includes a database that stores evaluation indices of the evaluation items for each of a plurality of rehabilitation patients regarding evaluation items for evaluating the level of achievement of the rehabilitation training. The display unit 202 displays information regarding a plurality of rehabilitation patients in order to assist the rehabilitation of a target patient. The assistance apparatus 200 divides a plurality of rehabilitation patients into groups in accordance with the evaluation index and displays the results of the division. Further, the display unit 202 updates the display that shows the state of progress of the target patient in accordance with the progress information of the rehabilitation training and shows the display. The data in the storage unit 203 is updated in accordance with the progress of the rehabilitation training performed by the rehabilitation patient. Accordingly, it is possible to assist the rehabilitation of the target patient more appropriately.

While the example of displaying the state at the time of discharge has been described in the aforementioned description, a state in which a predetermined number of days have passed since the date of the onset of symptoms may instead be displayed.

Further, a part or all of the processing in the aforementioned systems, apparatuses, terminals and the like may be provided as a computer program. The program can be stored and provided to a computer using any type of non-transitory computer readable media. Non-transitory computer readable media include any type of tangible storage media. Examples of non-transitory computer readable media include magnetic storage media (such as flexible disks, magnetic tapes, hard disk drives, etc.), optical magnetic storage media (e.g. magneto-optical disks), CD-ROM (Read Only Memory), CD-R, CD-R/W, and semiconductor memories (such as mask ROM, PROM (Programmable ROM), EPROM (Erasable PROM), flash ROM, RAM (Random Access Memory), etc.). The program may be provided to a computer using any type of transitory computer readable media. Examples of transitory computer readable media include electric signals, optical signals, and electromagnetic waves. Transitory computer readable media can provide the program to a computer via a wired communication line (e.g. electric wires, and optical fibers) or a wireless communication line.

Note that the present disclosure is not limited to the above embodiments and may be changed as appropriate without departing from the spirit of the present disclosure.

From the disclosure thus described, it will be obvious that the embodiments of the disclosure may be varied in many ways. Such variations are not to be regarded as a departure from the spirit and scope of the disclosure, and all such modifications as would be obvious to one skilled in the art are intended for inclusion within the scope of the following claims.

What is claimed is:

1. A rehabilitation assistance system comprising:
a database configured to store, for each rehabilitation patient, evaluation indices of evaluation items regarding the evaluation items for evaluating a level of achievement of rehabilitation training;
a processing unit configured to divide a plurality of rehabilitation patients into groups in accordance with the evaluation indices; and
a display unit configured to update a display that shows a state of progress of a target patient in one of the groups in accordance with progress information of rehabilitation training and show the display.

2. The rehabilitation assistance system according to claim 1, comprising displaying information on the number of persons indicating the number of rehabilitation patients who belong to the group as the display that shows the state of progress of the target patient.

3. The rehabilitation assistance system according to claim 2, wherein
two or three evaluation items among a plurality of evaluation items are set as priority evaluation items,
a plurality of rehabilitation patients who belong to the selected group are divided into subgroups in accordance with the evaluation indices of non-priority evaluation items other than the priority evaluation items, and
information on the number of persons indicating the number of rehabilitation patients who belong to each of the respective subgroups is displayed.

4. The rehabilitation assistance system according to claim 3, wherein
the number of priority evaluation items is two, and
the information on the number of persons of the group is displayed in a form of a two-dimensional matrix.

5. The rehabilitation assistance system according to claim 1, comprising filtering the plurality of rehabilitation patients according to a search condition in accordance with the target patient.

6. The rehabilitation assistance system according to claim 1, wherein the evaluation items include a FIM.

7. The rehabilitation assistance system according to claim 1, wherein the evaluation items include adaptive equipment worn by the rehabilitation patient.

8. The rehabilitation assistance system according to claim 1, wherein the evaluation items include an assistive device used by the rehabilitation patient.

9. A rehabilitation assistance method comprising:
referring to a database configured to store, for each rehabilitation patient, evaluation indices of evaluation items regarding the evaluation items for evaluating a level of achievement of rehabilitation training, and dividing a plurality of rehabilitation patients into groups; and
updating a display that shows a state of progress of a target patient in one of the groups in accordance with progress information of rehabilitation training and showing the display.

10. The rehabilitation assistance method according to claim 9, comprising displaying information on the number of persons indicating the number of rehabilitation patients who belong to the group as the display that shows the state of progress of the target patient.

11. The rehabilitation assistance method according to claim 10, wherein
two or three evaluation items among a plurality of evaluation items are set as priority evaluation items,
a plurality of rehabilitation patients who belong to the selected group are divided into subgroups in accordance with the evaluation indices of non-priority evaluation items other than the priority evaluation items, and
information on the number of persons indicating the number of rehabilitation patients who belong to each of the respective subgroups is displayed.

12. The rehabilitation assistance method according to claim 11, wherein
the number of priority evaluation items is two, and
the information on the number of persons of the group is displayed in a form of a two-dimensional matrix.

13. The rehabilitation assistance method according to claim 9, comprising filtering the plurality of rehabilitation patients according to a search condition in accordance with the target patient.

14. The rehabilitation assistance method according to claim 9, wherein the evaluation items include a FIM.

15. The rehabilitation assistance method according to claim 9, wherein the evaluation items include adaptive equipment worn by the rehabilitation patient.

16. The rehabilitation assistance method according to claim 9, wherein the evaluation items include an assistive device used by the rehabilitation patient.

17. A non-transitory computer readable medium storing a program for causing a computer to execute the rehabilitation assistance method according to claim 9.
